(19) United States
(12) Patent Application Publication
(10) Pub. No.: US 2020/0381119 A1
(43) Pub. Date: Dec. 3, 2020

(54) Veterinary Telemedicine System and Method

(71) Applicants: Andy H. Gibbs, Tucson, AZ (US); Ian D. Hochberg, Oro Valley, AZ (US)

(72) Inventors: Andy H. Gibbs, Tucson, AZ (US); Ian D. Hochberg, Oro Valley, AZ (US)

(21) Appl. No.: 16/885,106

(22) Filed: May 27, 2020

Related U.S. Application Data
(60) Provisional application No. 62/853,139, filed on May 27, 2019.

(51) Int. Cl. G16H 50/20; G16H 70/60; G16H 10/60; G16H 50/70; G16H 40/67; G16H 20/60; G16H 80/00; G06K 19/07; G16H 10/65; A61B 5/00; A61B 5/11

(52) U.S. Cl. CPC G16H 50/20; A61B 5/4803; G16H 10/60; G16H 50/70; G16H 40/67; G16H 20/60; G16H 80/00; G06K 19/0723; G16H 10/65; A61B 5/0022; A61B 5/7267; A61B 5/7246; A61B 5/7275; A61B 5/6822; A61B 5/6887; A61B 5/0077; A61B 5/4866; A61B 5/7282; A61B 5/7465; A61B 5/1118; A61B 2503/40; A61B 2562/0204; A61B 2562/08; A61B 2562/0219; A61B 2562/0223; G16H 70/60

(57) ABSTRACT

A veterinary telemedicine system and method for remote diagnosis of pets. The veterinary telemedicine system and method generally includes a library of pet behaviors associated with an individual pet, each behavior comprising a data signature associated with the behavior. The system includes a local computer, which may be a smartphone, adapted to receive a set of data from a plurality of sensors and further adapted to transmit the set of data to a cloud database. The system also includes a smart collar wearable by a pet and comprising a sensor adapted to track a behavior of the pet and further adapted to communicate with the local computer to provide the local computer with pet behavior data from the sensor. The library of pet behaviors is stored on the cloud database and is usable to diagnose pet health via machine learning/artificial intelligence.

### CROSS REFERENCE TO RELATED APPLICATIONS

I hereby claim benefit under Title 35, United States Code, Section 119(e) of U.S. provisional patent application Ser. No. 62/853,139 filed May 27, 2019. The 62/853,139 application is currently pending. The 62/853,139 application is hereby incorporated by reference into this application.

### STATEMENT REGARDING FEDERALLY SPONSORED RESEARCH OR DEVELOPMENT

Not applicable to this application.

### BACKGROUND

### Field

The novel companion animal informatics system is directed toward the field of veterinary telehealth.

### Related Art

Any discussion of the related art throughout the specification should in no way be considered as an admission that such related art is widely known or forms part of common general knowledge in the field.

The American Veterinary Medical Association (“AVMA”) defines “telehealth” as the overarching term encompassing various digitally enabled took including “mHealth”, “telemedicine”, “teleconsulting”, “telemonitoring” and “telemedicine”, each a distinctly different technology used by a veterinarian to remotely deliver pet health information or care.

More specifically, the AVMA defines mHealth, also referred to as mobile health, as telehealth subcategory employing mobile devices such a smartphones, and describes the telemonitoring subcategory as the remote monitoring of animal patients that are not at the same location as the health care provider, including for example, a wearable monitoring device that captures the animal's vital signs and other behaviors.

Further, it defines “telemedicine” as a subcategory of telehealth that involves use of a tool to exchange medical information electronically from one site to another to improve a patient's clinical health status; telemedicine is tool of practice, not a veterinary discipline. Telemedicine facilitates communication, diagnostics, treatments and other tasks. Currently, telemedicine is typically used, for example by a general practice veterinarian to digitally transmit patient data such as X-Rays or CT scans to a veterinary specialist.

Many in the pet industry incorrectly conflate mHealth solutions to be telemedicine solutions. However, telemedicine solutions that employ video, voice and or text communication between a pet owner and remotely located veterinarian but lack the delivery of diagnostic, clinical or pet behavioral data need by the veterinarian for use in diagnosis or treatment of a condition fail to meet the AVMA's definition of telemedicine.

Those skilled in the art will appreciate both the economic and pet health care value of a novel telemedicine solution that incorporates the in-home telemonitoring of a pet, combined with the in-home acquisition, storage and analysis of clinical, diagnostic and/or behavioral data associated with an individual pet along with the pet's lifetime digital health history, all of the clinical, diagnostic or behavioral data further communicated via a telemedicine connection between the pet's owner and remotely located veterinarian who could then deliver the fastest, lowest cost, and most reliable diagnosis and treatment.

### SUMMARY

The novel telemedicine system comprises one or more pet telemonitoring devices that acquire data associated with each individual pet, communicates the data to a central database on a network, uses rules-based analysis of the data to identify real-time, historical, computed pet behaviors, behavior trends and trend anomalies, applies machine learning and artificial intelligence to trend anomalies to predictively diagnose the underlying cause of the anomalies, and proactively communicates the current, historical, and anomalous data, along with predictive diagnostics information to a remotely located veterinarian. The proactive telemedicine system therefore provides the pet health data necessary for improving a veterinarian's virtual examination of the pet, reduces mis-diagnoses, and allows veterinarians to more accurately prescribe treatment protocols based on the pet's historical, clinical and predictive diagnostic information.

There has thus been outlined, rather broadly, some of the embodiments of the veterinary telemedicine system and method in order that the detailed description thereof may be better understood, and in order that the present contribution to the art may be better appreciated. There are additional embodiments of the veterinary telemedicine system and method that will be described hereinafter and that will form the subject matter of the claims appended hereto. In this respect, before explaining at least one embodiment of the veterinary telemedicine system and method in detail, it is to be understood that the veterinary telemedicine system and method is not limited in its application to the details of construction or to the arrangements of the components set forth in the following description or illustrated in the drawings. The veterinary telemedicine system and method is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Example embodiments will become more fully understood from the detailed description given herein below and the accompanying drawings, wherein like elements are represented by like reference characters, which are given by way of illustration only and thus are not limitative of the example embodiments herein.

FIG. 1 is an exemplary diagram showing various methods of identifying symptoms pet illness.

FIG. 2 is an exemplary diagram showing a flow chart of machine identification of animal illness symptoms.

FIG. 3 is an exemplary diagram showing a plurality of functional outcomes from prescriptive analysis of pet behavioral data.

FIG. 4 is an exemplary diagram showing a veterinary telemedicine system for data acquisition, analysis and display.

FIG. 5 is an exemplary diagram showing a chart of various kinds of data of a veterinary telemedicine system.

FIG. 6 is an exemplary diagram showing a representative data stack of a veterinary telemedicine system.

FIG. 7 is an exemplary diagram showing a process of data input, processing and creating actionable data output of a veterinary telemedicine system.

FIG. 8 is an exemplary diagram showing representative attributes of an animal hydration symptomology expert system.

FIG. 9 is an exemplary diagram showing examples of symptomology derived from data corresponding to nuanced animal behaviors.

FIG. 10 is an exemplary diagram showing a system and method of creating discrete digital signatures an animal behavior.

FIG. 11 is an exemplary diagram showing a plurality of digital signatures correlating to animal behaviors.

FIG. 12 is an exemplary diagram showing a data trend of one behavior for one animal compared to the trend of a larger cohort of similar animals.

FIG. 13 is an exemplary diagram showing the data trends relating to a plurality of behaviors exhibited by one animal over a period of time.

FIG. 14 is an exemplary diagram showing the improved telemedicine platform that connects the veterinarian, client and pet.

FIG. 15 is an exemplary diagram showing the predictive diagnosis of a disease derived from the analysis of a plurality of animal behaviors.

FIG. 16 is an exemplary diagram showing a method of remotely evaluating the efficacy of a treatment of an illness derived from the analysis of a plurality of animal behaviors.

FIG. 17 is an exemplary diagram showing a list of animal disorders and examples of the corresponding data acquisition means used to predictively identify the disorders.

FIG. 18 is an exemplary diagram showing a portion of an expert system.

### DETAILED DESCRIPTION

Various aspects of specific embodiments are disclosed in the following description and related drawings. Alternate embodiments may be devised without departing from the spirit or the scope of the present disclosure. Additionally, well-known elements of exemplary embodiments will not be described in detail or will be omitted so as not to obscure relevant details. Further, to facilitate an understanding of the description, a discussion of several terms used herein follows.

The word “exemplary” is used herein to mean “serving as an example, instance, or illustration.” Any embodiment described herein as “exemplary” is not necessarily to be construed as preferred or advantageous over other embodiments.

The word “animal” is used herein to mean a “companion animal”, “pet”, “dog”, or “cat”, and the use of any of these words should be interpreted as having the same meaning without limitation.

The word “sensor” is used herein to mean any active or passive device used to acquire data associated with a unique animal and without limitation may be analog or digital, and may include accelerometers, magnetometers, gyroscopes, load or pressure sensors, liquid level sensors, proximity sensors, motion sensors, sound sensors, RFID or NFC tags, temperature sensors, or a combination of sensors. Further, sensors may be incorporated into wearables such as a pet collar, or may be fixed within a pet's domicile in a permanent or semi-permanent location.

The phrase “behavior” is used herein to mean a set of data that is statistically and reliably similar to a previously defined set of data correlating to a specific movement or combination of movements, an activity, a physiological condition at a point in time, a change in physiological condition, a preference as demonstrated by a pet's generation of a repeatable data set, the statistically significant deviation from a previously repeatable data set, or a combination thereof. The data used to identify and/or define a behavior may be acquired from one or more sensors associated with a given pet, from data manually input by a pet guardian in response to a form questionnaire, from an external data source, for instance, an application program interface (API) that provides the historical, current and predicted weather conditions locally relevant to each given pet, computed data such as a dog's computed current age relative to the dog's date of birth, or any combination of these data or other data as may be used in defining a data set that correlates to the behavior.

Broadly, and without limitation, examples of a dog's behavior may include a data set indicative of the dog sleeping, running or licking, and for the duration that the data set occurs, for instance, sleeping for three hours, or running for five minutes, the data set being recorded on a computer media representing the continually updatable behavioral timeline of the pet throughout their lifetime. Further, a behavior may be an anomalous deviation from the historical trend, or the predicted continuation of that historical trend of any given behavior of a given pet, or a deviation of the pet's behavior when compared to the behavioral trends of a larger cohort of similar animals, the anomalous data therefore being interpreted as a desirable or undesirable behavior being exhibited by the pet. For instance, an anomalous, ever-increasing volume of daily water consumption over a given period of time may be indicative of a behavior that correlates to the onset of diabetes, a disease determined to be an undesirable behavior.

A behavior may therefore be identified by one or more of retrospective analysis of historical data, the near real-time occurrence of a data set, or may be predictive of a future behavior.

“Motion Capture” is used herein as one means of correlating a set of data with a given behavior. One method of understanding behaviors of animals in remote locations is to create a library of data sets indicative of specific behaviors, the library of data sets preferably created in a controlled laboratory environment wherein the data related to a given behavior can be accurately acquired and correlated. For instance, motion capture of a dog scratching its left ear, correlated to a simultaneously acquired data set obtained from a wearable sensor during the scratching episode would thereafter be cataloged as a “scratching left ear” behavior in a library of predefined animal behaviors. Motion capture therefore may preferably incorporate without limitation the use of one or more optical systems incorporating passive or active markers, non-optical systems incorporating inertia, mechanical motion or magnetic systems, or a combination of these systems to create a data representation of each discrete two or three dimensional behavior without limitation.

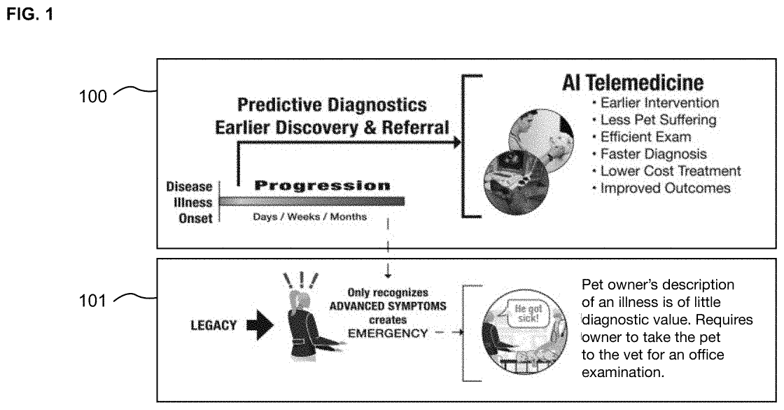

FIG. 1 is an exemplary diagram showing various methods of identifying symptoms pet illness. More specifically, as it has been well established since the founding of modern veterinary medicine during the first half of the 1800s, that the reactive ill pet referral system 101 relied on an animal's caretaker to observe an animal exhibiting unexpected symptoms that represented an underlying cause. Upon concluding that the animal was experiencing a health problem, the caretaker would transport the animal to a veterinarian for an office examination, illness diagnosis and perhaps, treatment. The fundamental problems of this system, which continue today, are that (1) pets notoriously hide symptoms until the underlying disease, illness or injury progresses to an advanced stage so as to be recognized as a problem by a typical pet guardian who lacks veterinary training; this ensures that the pet has endured suffering unnecessarily until the guardian finally recognized the symptoms, (2) relies on the guardian to describe changes in the pet's behavior prior to the onset of obvious symptoms; this produces a completely unreliable recollection of the change in the pet's activity, eating or drinking behavior since these changes occurred before the guardian recognized the advanced symptoms, (3) the delayed recognition of the first onset of the pet's health decline creates a higher level of urgency in seeking veterinary care, increases costs of care, and yields a poorer prognosis compared to earlier intervention by the veterinarian.

On the other hand, a data-driven veterinary telemedicine system provides for continuous analysis of even minor changes in a pet's activity, eating, drinking, or other tell-tale signs synonymous with an underlying cause such as an illness, advancing chronic disease or injury, and proactively alerts the guardian and/or proactively connects the pet with a remotely located veterinarian who can review the historical and current pet health data to diagnose and prescribe earlier treatment that reduces pet suffering, reduces treatment costs paid by the guardian or a pet health insurance provider, and accelerates recovery, none of which can be provided by the legacy reactive ill pet referral system first described.

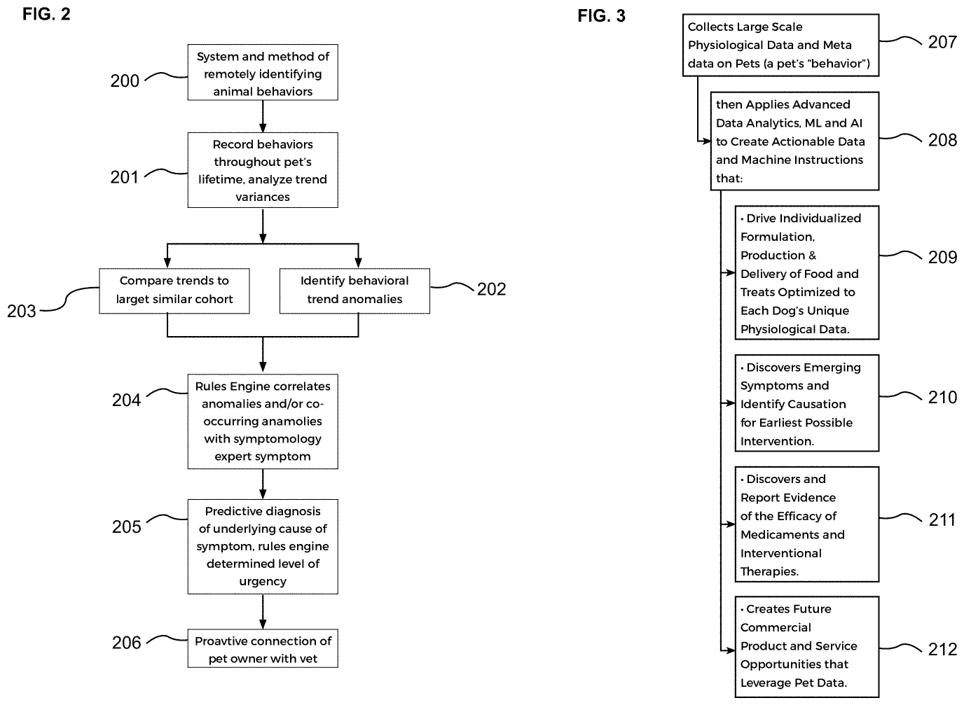

FIG. 2 is an exemplary diagram showing a flow chart of machine identification of animal illness symptoms. The preferred functional elements of an illness identification and veterinary care referral system, which will be described in more detail below, generally comprise a system of identifying pet behaviors 200, a method of digitally recording pet behaviors 201 on a telemedicine system, a machine analysis that may identify anomalous trends in a pet's behavior 202, a machine-performed comparison of the current pet's behavior anomalies to the behaviors of a similar cohort of pets 203, a machine method of comparing the behavior anomalies to a symptomology expert system 204, the machine generation of a list of possible causes and urgency medical that exhibit similar symptomology 205, and a machine method of proactively connecting the ill pet with a veterinary care provider 206.

FIG. 3 is an exemplary diagram showing a plurality of functional outcomes from prescriptive analysis of pet behavioral data. Large scale data acquired from multiple sources, all of the data being associated with a specific pet, provides virtually unlimited opportunities to analyze the data to identify a condition, want, need or preference of the specific pet at any point in time. The analysis of the pet data may incorporate machine learning and artificial intelligence as a means to learn more, and become increasingly more accurate over time in predicting the pet's wants, needs and preferences.

In the drawing, large scale data and meta data are preferably acquired from a plurality of sources not shown, but will be fully described herein, the data used to identify a pet's behavior 207. Using rules based machine learning and artificial intelligence 208, each pet's behavior is analyzed to identify the unique wants or needs at any given point in time. For instance, a behavior data set that establishes the predicted daily caloric requirements of an 8-year old dog would guide the machine recommendation 209 of an appropriate food and treat type, the daily portions, and the food re-ordering schedule based on the size of a food package and predicted and/or actual consumption. In another instance, the analysis of a pet's behavior, and more specifically the change and rate of change in one or more behaviors may be indicative of a health problem 210. In another instance in which a veterinarian prescribes a medicament to treat a medical problem, the analysis of behavior before and after the pet begins a prescription regimen provide the veterinarian with a data-supported understanding of the efficacy of the medicament 211. The behavior data analysis further provides for various commercial opportunities to provide uniquely tailored products or services to the pet. Merely presented as one example of many behavior data driven commercial opportunities 212, current and historical data indicative of a generally healthy pet lifestyle may be of commercial value to pet health insurance desiring to provide lower premium insurance to healthier animals that are will have fewer and lower cost insurance claims when compared to animals with a data history living unhealthy lifestyles.

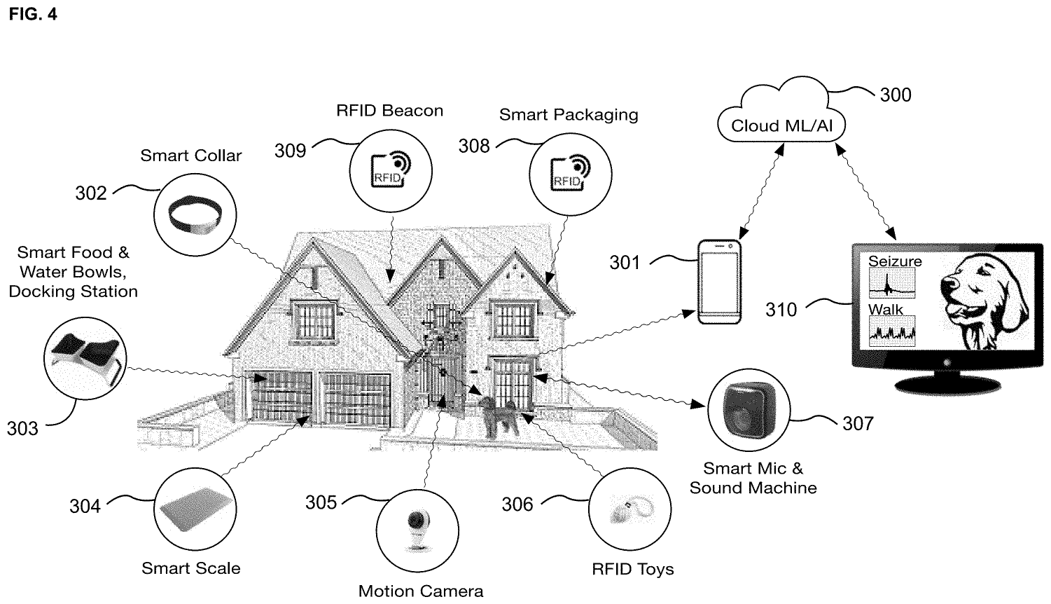

FIG. 4 is an exemplary diagram showing a veterinary telemedicine system for data acquisition, analysis and display. An Internet of Things (“IoT”) telemonitoring system comprises one or more sensors or devices incorporating sensors that transmit the collected data to a cloud database via a local WiFi router and/or Bluetooth connectivity via a software application installed on a smartphone or other mobile device. Once the pet data is recorded to the pet's unique pet profile in the cloud, software processes it and then might decide to perform an action, such as sending an alert or automatically adjusting the sensors/devices without the need for the user.

More specifically, a smart collar 302 provides for sensors that track and record the movements of an animal in and around their living environment. Additional data used in analyzing each pet may be acquired from pressure sensors integrated into a water and food bowl 303, a weighing scale 304, a motion sensing camera 305, toys 306 containing a unique RFID or NFC tag used to determine proximity to or use by a pet, a sound acquisition device such as a smart microphone and speaker 307, food, drug or other consumable packaging 308 identified by a unique RFID tag, and proximity sensors such as an RFID beacon 309 that sense the track a pet's movement throughout their home, the components of the IoT system just described are not intended to be limiting. Data acquired from the one or more IoT devices is communicated to a cloud database 300 via a WiFi router proximate to the pet's living environment, or via a software app installed on a smartphone 301.

The pet's data is available to a veterinarian via a telemedicine platform that may simultaneously display real time video of the pet along with historical, current, real-time near real-time, or predicted diagnostic data on a computer via an internet-accessible telemedicine portal 310.

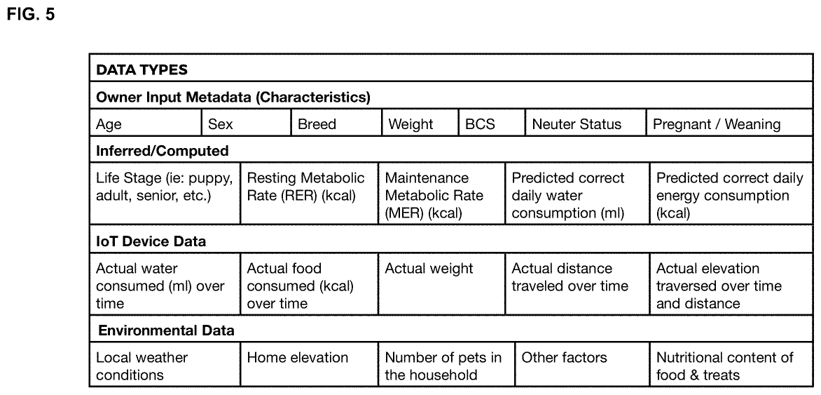

FIG. 5 is an exemplary diagram showing a chart of various kinds of data of a veterinary telemedicine system. It is well known that the more information that is available regarding each pet, the more comprehensive and more accurate the analysis of the pet's behavior. The scope of data related to any individual pet is virtually endless, so the following examples are not meant to be limiting. Listing every type of acquired, computed or derived data would be unduly cumbersome, yet to describe every possible type of data contributing to the determination of each pet's behavior would reinforce the importance of acquiring the maximum data from the largest number of sources to increase accuracy of remotely identifying pet behaviors.

In the chart, owner input data associated with their pet may include age, sex, breed, weight, body fat percentage referred to a Body Condition Score (“BCS”), and other unique physiological attributes associated with that pet. Importantly, some of this owner input data will change over a pet's lifetime, for instance age and weight, while other data will not change, for instance, the pet's breed and sex. It is necessary to incorporate rules into a telemedicine system to analyze current behaviors based on both the static and dynamic data.

Inferred or computed data may include the computed life stage based on breed and age of the pet. Industry experts consider pets to transition through four life stages: growth (i.e. puppy), adult, senior and geriatric. Each life stage brings with it, among many other things, differences in nutritional requirements, and actual versus predicted energy requirements that typically correspond to different levels of activity. It is important to note that large breed dogs such as Great Danes transition from growth to geriatric in 5-7 years, while small breed dogs may transition through the life stages over 15 or more years. The computed life stage data must acknowledge well-known differenced based on breed, sex and other manually data input during the pet profile setup.

As previously discussed, data may be received from one or more IoT devices, the data continually communicated to the cloud database thereby updating the behavioral data, while building the historical pet data. The IoT data may be continually analyzed to develop behavioral trends, and to assess the trends for anomalous changes that may indicate an important change in the pet's condition.

Third party data sources play an important part in analyzing pet behavior, and more importantly, anomalous trends in the pet's behavior that may indicate a health problem. For instance, the chart shows local weather conditions that may be loaded into the pet's profile on the cloud via a weather API. When running around during warm weather, a pet is expected to drink considerably more water than when cooped up in the house during the winter. Absent the local weather data, an increase in drinking may be incorrectly identified as a behavior synonymous with a medical condition known as Polydipsia—an increase in water consumption caused by a physiological or psychological problem that would require medical care.

The data types and sources just presented represent merely a small sampling of the data types and sources of the preferred veterinary telemedicine system, and are not meant to be limiting.

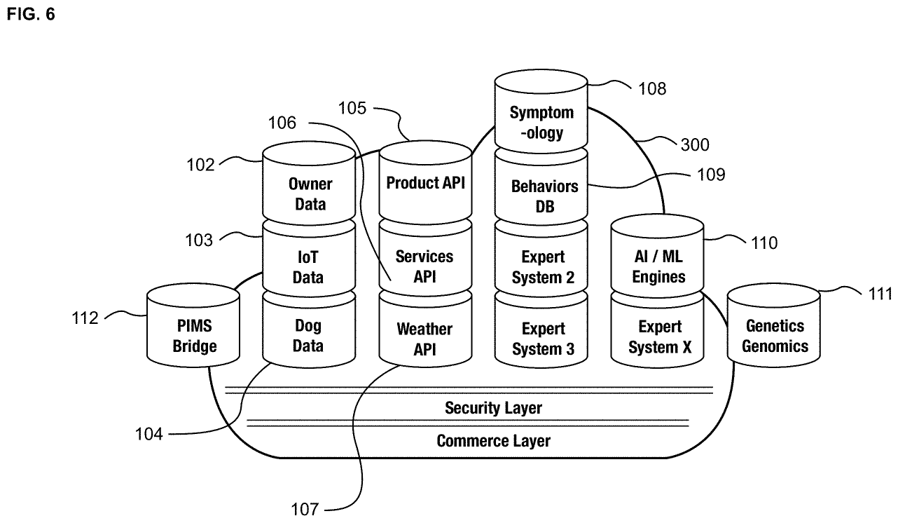

FIG. 6 is an exemplary diagram showing a representative data stack of a veterinary telemedicine system. The architecture of a cloud database 300 comprises an owner database 102 containing personally identifiable data on each pet owner, an IoT database 103 containing data received from a plurality of IoT devices deployed in the field, and a database containing personally identifiable pet profiles 104. Further, a data stack provides for various expert systems which may include lookup tables as may be required for pet behavior analysis, the expert systems including but not limited to a symptomology expert system 108 that contains the typical symptoms encountered with a virtually limitless list of pet illnesses, diseases and injuries, and a behaviors database 109 containing a virtually limitless number of data sets each specifically correlating to a previously determined behavior. For instance, the data stream received from an IoT collar during the period that a dog is drinking water will be cataloged as a “dog drinking” data signature. Still further, the data stack includes a plurality of rules-based engines 110 that support and include machine learning and artificial intelligence, and various APIs, third party data feeds that preferably include a pet products API 105, a pet services API 106, a weather conditions API 107, and other APIs as may support or broaden the functionality of the telemedicine platform. An animal genetics/genomics database 111 provides for expanded predictive health analysis of each pet based on breed and other factors well known to those skilled in the art, and further provides a bridge 112 to veterinarians' practice information management systems (“PIMS”) as a means of securely transmitting the owner and pet data of the telemedicine system into the veterinarians own client/patient records.

The symptomology expert system is nearly encyclopedic in nature because of the depth and breadth of symptoms presenting from many hundreds of pet illnesses, injuries and diseases, and is manually created by experts in veterinary symptomology and/or emergency care triage.

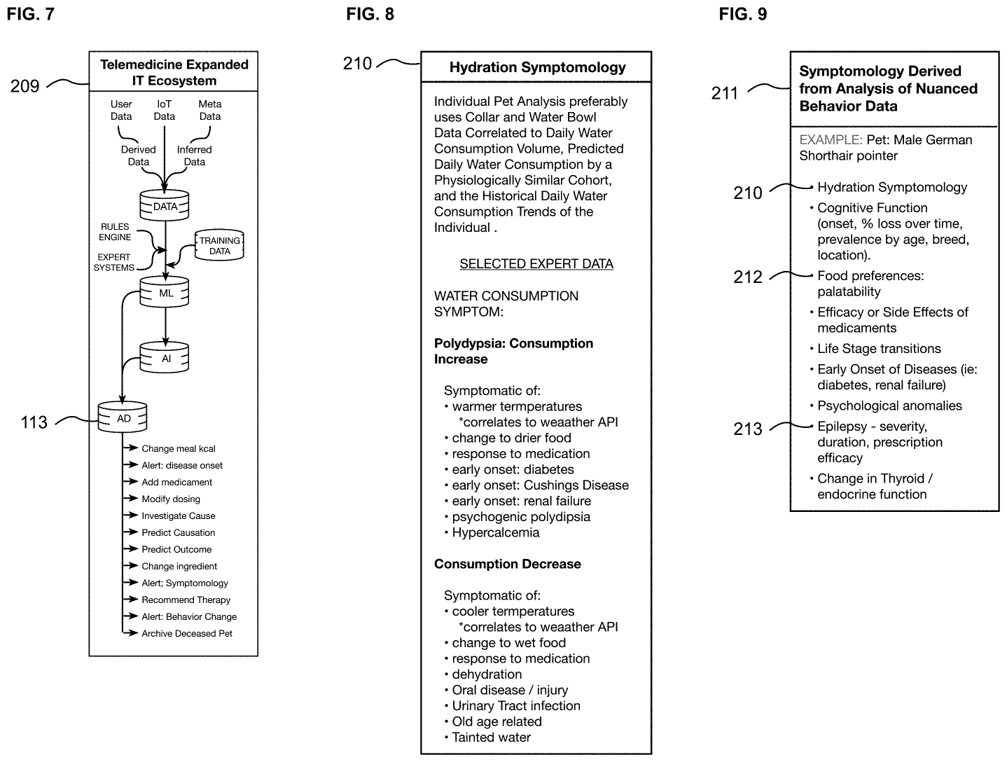

FIG. 7 is an exemplary diagram showing a process of data input, processing and creating actionable data output of a veterinary telemedicine system. As just described, a telemedicine platform acquires pet related data from a plurality of sources including data and metadata computed, derived and/or associated with the data. Rules-based engines are used to process and analyze the data, including machine learning (“ML”) and artificial intelligence (“AI”) which correlate pet data and trends with pet wants and needs, and further which identify data anomalies indicative of a health issue. AI provides decision-support that drives actionable data 113 as a functional output of the telemedicine system, the actionable data including autonomous decisions such as modifying the daily caloric requirements of any given pet based on weight, age and other factors, and/or generate and sent a health alert by text, email, or via a smartphone app or web portal, to the pet owner and/or veterinarian when AI predicts the onset of an illness or disease based on undesirable deviations from the pet's predicted or real-time behavior profile. The list of actionable data driven functions shown provides a glimpse of decisions made by a proactive telemedicine system, the list of functions therefore is not meant to be limiting.

FIG. 8 is an exemplary diagram showing representative attributes of an animal hydration symptomology expert system. As described (U.S. application Ser. No. 12/987,080, FIGS. 20-31) proper hydration is critical to long-term pet health. Recognizing the over-consumption or under-consumption of water has long been used by veterinary professionals as a diagnostic method to identify health problems.

As one example of computing the predictive diagnosis based on analyzing data associated with each pet, daily water consumption is tracked by means of a smart water bowl that records total water consumed from the bowl, and/or by means of correlating the duration of multiple drinking behavior signatures received during the day from a smart collar. The data represents the total water consumed during the day, and the daily consumption data is appended to the pet's profile record. ML continually analyzes the data to maintain water consumption trends for each pet, and may identify increases or decreases in water consumption that are out of range from the predicted high/low range based on the pet's historical trend. As described above, the rules-based ML, upon identifying anomalous drinking data or drinking trends that stray out of a predetermined acceptable range trigger a lookup on a symptomology expert system. In the chart, if the water consumption anomaly is an increase, underlying causes associated with an increase in water consumption are identified. It would be impractical to assume that all of the possible causes would apply to any given pet, so a triage of potential causes is preferably performed by artificial intelligence employing predefined rules, or learned rules unique to each pet. For instance, the chart shows that an increase in temperature at the pet's location may account for the increase in water consumption. Using rules-based triage and lookup of the daily temperature trends as delivered via a weather API, the machine will determine if temperature increase is a likely cause, and include or exclude temperature change as a possible underlying cause for the increased water consumption.

Further, each of the underlying causes shown, for example: change to drier food, response to medication, early onset: diabetes, early onset: Cushings Disease, early onset: renal failure, psychogenic polydipsia, or hypercalcemia are further correlated to the level of medical urgency which drive the autonomous notifications to pet owners or veterinarians, the notification process not being shown in the present chart, but which were previously discusses.

As will be appreciated, the symptomology expert system is substantially broad and deep, containing hundreds to thousands of different pet illnesses, diseases and injuries along with typically associated symptoms and causes, all of which comprise the symptomology expert system urgency analytics and autonomous notification process.

FIG. 9 is an exemplary diagram showing examples of symptomology derived from data corresponding to nuanced animal behaviors. In the chart 211, hydration symptomology 210 which was just described in FIG. 8 is shown again as but one of the many hundreds of illnesses, diseases, injuries or conditions of a pet. Robust rules engines provide for continual analysis of the large-scale data associated with each pet, the rules each analyzing behaviors to identify or discover trend or acute data anomalies, and associate those anomalies with possible underlying causes. As an example of another of the many behavioral trends analyzed, food palatability 212 identifies the food each pet desires most. The analysis of data acquired from the IoT food bowl provides the computed duration of time it takes for a dog to consume its meal. Faster consumption times have proven to be synonymous with a preferred food compared to an alternate food. A-B testing between two different foods is a standard pet food industry practice to identify one higher palatability, tastier food compared to another that the pet takes longer to consume. (palatability previously disclosed GIBB-070, FIGS. 85-93)

Epilepsy 213 is the number one neurological disorder in dogs, yet one of the least diagnosed. Diagnosis first requires the pet owner to observe a seizure before contacting a veterinarian. Oftentimes, an epileptic seizure will occur at night while the owners are sleeping, or during the day when owners are away at work, hence, the seizure is not observed. The present telemedicine system would map the anomalous data stream sent during a seizure against the behavior database previously described in FIG. 6, against the library of signatures to identify the anomalous data stream as a seizure, and record the actual seizure data in the pet's personal profile. The symptomology ML would add the newly discovered seizure to the library of epileptic seizures to expand its knowledge base of the different types of seizures. A seizure would trigger an autonomous notice to the pet owner regarding the seizure, and make available to the veterinarian the seizure data to aid in diagnosis and prescribed treatment relative to the intensity, severity, duration and recovery time of the seizure.

As will be appreciated, the veterinary telemedicine platform just described provides predictive diagnostics and historical pet data previously unavailable with any known commercially available veterinary telemedicine system.

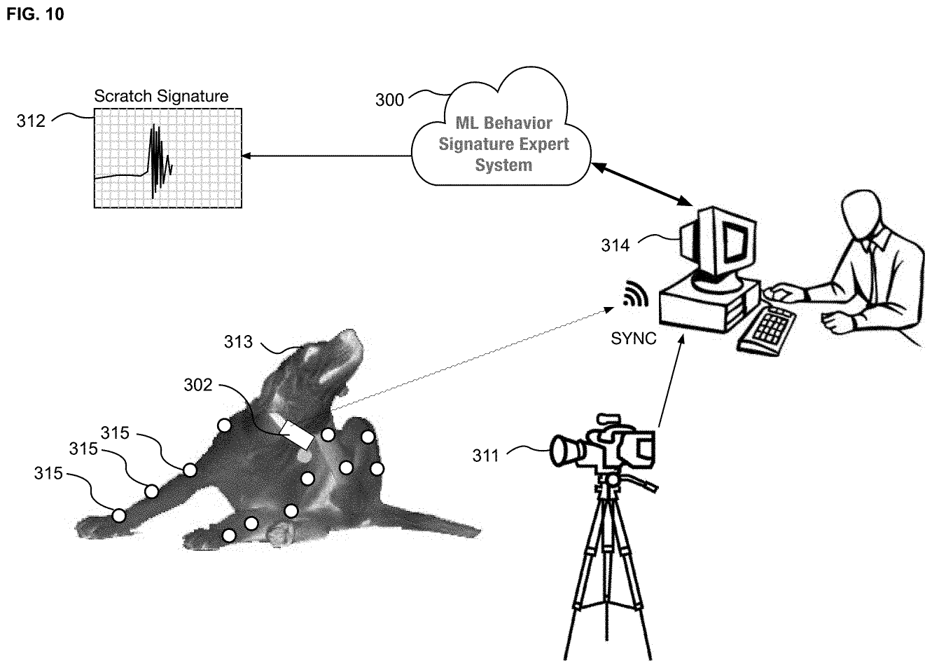

FIG. 10 is an exemplary diagram showing a system and method of creating discrete digital signatures an animal behavior. As previously described, the telemedicine system comprises a behavior database (FIG. 6, 109) containing data sets mapped to innumerable and uniquely identifiable behaviors such as a dog walking versus running versus trotting. The process of creating and training the behavior database is laborious, and the refinement of the behavior database to include even nuanced behaviors is ongoing. For instance, the data stream sent by a smart collar while a dog is walking will be very similar to a dog walking with a limp, and only continuous training and analysis of a data set associated with a limp compared against the dog's historical data set associated with walking normally will be identified as a limp.

One preferred method of ensuring the accuracy of a machine to remotely identify a limp, or any other nuanced behavioral anomaly, absent the ability of a human to observe and confirm the correlation of the data set to the animal limping, is to build the behavior database using motion capture technology within a controlled environment.

In the drawing, a video camera is shown observing a dog 313 scratching. Preferably, a plurality of cameras would be used to capture the motion from multiple angles, the multiple camera data providing the information necessary to build a 3D model of the scratching dog. The methods of creating 3D models from living subjects are well known in the gaming and film industry. A plurality of markers 305 are temporarily fixed to various location on the dog, the locations preferably being adjacent to major joints, tail, head, and at certain points along the back and sides, the markers providing points that can be mapped on the video as the video is recorded on a computer 314. At the same time, data is sent from the smart collar 302 to the computer to be synced to the video, thereby providing a digitized model of the scratching dog, movements of each of the joints during scratching, and further, the data set received from the collar during the scratching event. Having been first-person observed, the computer operator manually tags the data set, which now includes the digitized video, as a scratching signature 312 and records the identified signature in the behavior database in the cloud 300.

It will be appreciated that nuanced differences will be exhibited between the data set acquired when the dog first scratches its left side compared to the right side based on the accelerometer and/or magnetometer data. These nuanced differences will be further annotated by the computer operator and recorded in the behavior library on the cloud.

Further, it will be appreciated that small dogs may scratch at faster frequencies than large dogs. While the absolute differences in the data sets will be obvious, the ML will increasingly refine the accuracy of scratching pattern recognition as the cohort of scratching dogs are analyzed using motion capture.

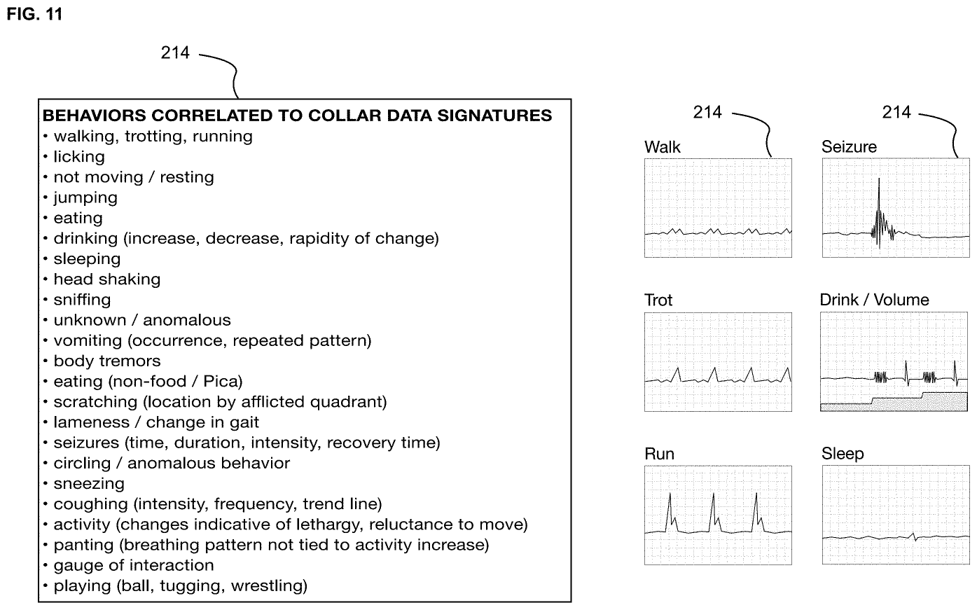

FIG. 11 is an exemplary diagram showing a plurality of digital signatures correlating to animal behaviors. The behaviors signature chart 214 provides a partial listing of behaviors that correlate to data sets, referred to herein as behavior signatures, each signature having been created and catalogued using the previously described motion and collar data analysis. Causation of each behavior is not necessarily known, as the diagnosis of the predicted cause of each behavior requires analysis against a symptomology database previously described, but not shown.

Merely for illustration purposes, six separate behavior signatures 214 are shown signifying the differences in the data sets, the differences preferably including the start and stop time of the behavior from which the duration is computed, the amplitude and frequency of the behavior data, and the period of time between each occurrence of the behavior, for instance, a dog that scratches for 30 seconds every 5 minutes versus a dos that scratches for 10-15 seconds a few times per day. These differences are used to determine whether the scratching trend is normal, or indicative of a possible health problem.

As previously described, ML will conduct behavior pattern matching between a remotely located animal and the behavior database, recognizing that variances in duration, frequency, amplitude and other characteristics unique to each dog will occur and not perfectly match the reference signature. However, being trained to recognize patterns, ML will increase accuracy of recognizing data set variances of each behavior by adding newly acquired behavior data sets to its behavior library.

Further, trend analysis on the patterns of each behavior will provide a statistically reliable norm against which anomalous patterns of the same behavior type can be identified.

Behavior signatures are not only acquired from smart collar data, but from other IoT devices as will be described more fully below.

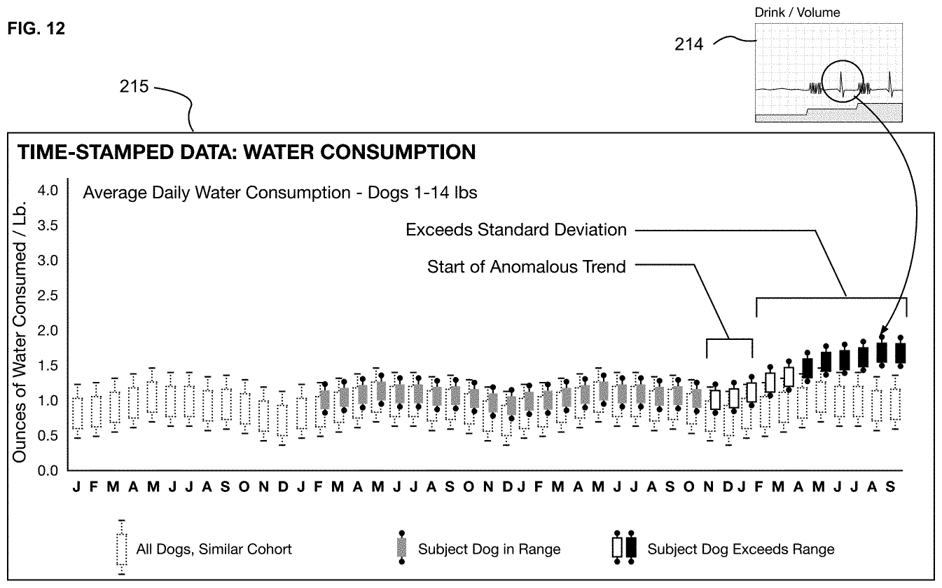

FIG. 12 is an exemplary diagram showing a data trend of one behavior for one animal compared to the trend of a larger cohort of similar animals. More specifically, a chart for water consumption behavior 215 shows minimum-maximum daily water consumption of a similar cohort of dogs weighing 1-14 pounds over many months, although data views of behavior trends may consist of any desirable time frame. In the chart, it is readily recognizable that average monthly water consumption increases during warm summer months, and decreases during cooler months. The pattern for this cohort predictably repeats itself year over year, but is self-correcting based on the min-max, mean and/or average water consumption of this cohort over time.

A subject dog is added to the database during a month of February as can be seen in the shaded candle markers. For a time, the subject dog consumes water volumes each month consistent with the larger cohort consumption behavior. However, it can be seen when the shaded candle turn white that the subject dog's monthly water consumption averages are increasing relative to the larger cohort, as well as increasing relative to its own historical water consumption behavior. At this point when the subject dog's pattern deviates from the cohort norm, but still remains within the outside tolerances of the cohort, the ML of the telemedicine platform triggers a notice to the pet owner that an appearance of a change in drinking behavior has occurred. Further, the subject dog's drinking behavior exceeds the monthly maximum averages for the cohort, triggering a warning notice indicating an anomaly.

Further, ML analyzes the anomaly, and polls the symptomology database to identify illnesses or diseases that may manifest in gradual but constantly increasing water consumption. For instance, this behavior pattern would positively correlate to early onset of renal failure or diabetes, both of which would require prompt veterinary diagnosis and treatment.

Recognition of the behavior-disease correlation of the subject dog's behavior, AI determines the appropriate action, and triggers the health notice to the pet owner, along with the possible level of urgency with which to seek veterinary care, and may further autonomously send the pet's predicted diagnosis to the pet's veterinarian via text, email or the telemedicine portal. The data of the chart just described is made available to the veterinarian to aid in further diagnosis and development of a treatment protocol.

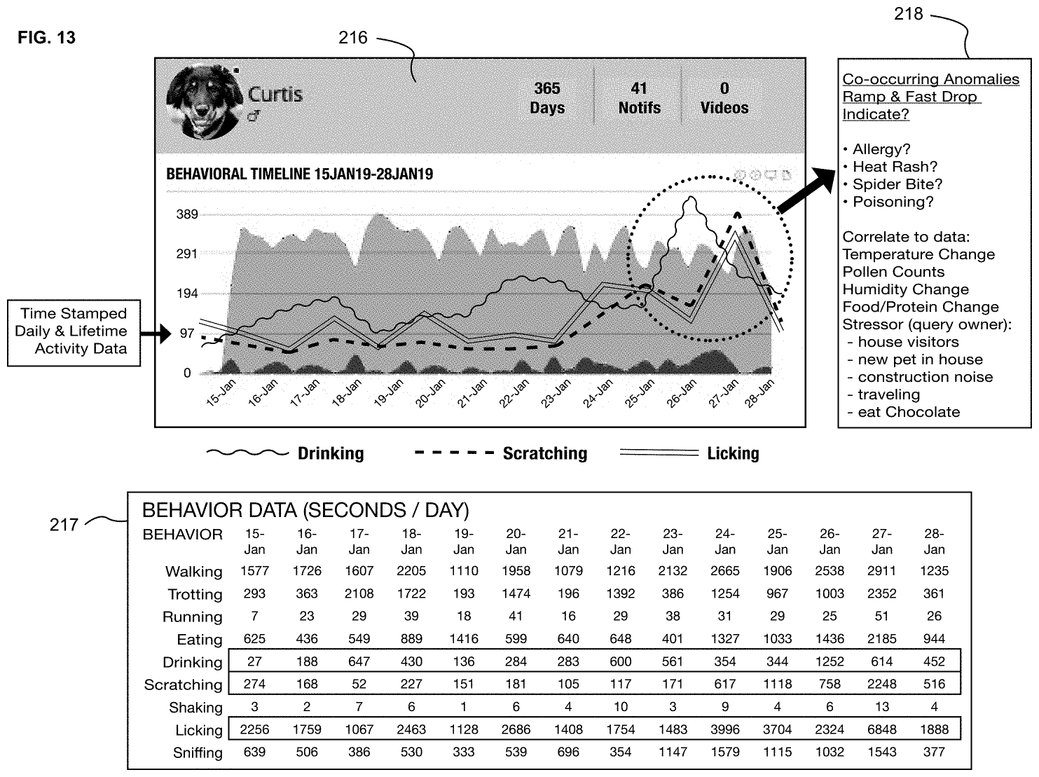

FIG. 13 is an exemplary diagram showing the data trends relating to a plurality of behaviors exhibited by one animal over a period of time. The accuracy of a predicted diagnosis such as the renal failure or diabetes just described is increased considerably when co-occurring behavior anomalies are also considered.

A daily behaviors graph 216 for a dog named Curtis presents a visual representation of 9 different behaviors which are separately listed below the graph along with daily behaviors data scores 217. As can be readily seen, a notable spike in three separate behaviors occurred during the most recent 3 days, notably a marked increase in water consumption, along with an increase in scratching and itching, anomalous when analyzed against Curtis' prior 10 days.

Evaluated alone, the water consumption behavior increase as just described may correlate to an underlying cause of diabetes. However, considering the anomalous co-occurring behaviors of increased scratching, both of which are dispositive to the inset of diabetes, eliminate diabetes and a possible underlying cause. Taken together, ML polls a symptomology database for possible illnesses, injuries or diseases that (1) have a rapid onset, and (2) generally cause co-occurring symptoms as presented in Curtis' chart.

Co-occurring symptomology 218 of increased scratching, licking and water consumption produces a short list of possible causes including, for example, the onset of an allergy, heat rash, an insect bite or poisoning.

Recognizing the possible causes, AI triggers notices to be autonomously sent to the pet owner and/or veterinarian along with the recent history representing the anomalous spikes in certain behaviors. The data therefore provides considerable understanding by the veterinarian of the onset, intensity and duration of the anomalous behavior from which the veterinarian can quickly perform further diagnosis to determine the actual cause of the behavioral change.

Further, as can be seen below the short list of possible causes, ML can further triage the possible causes by analyzing a weather API to detect possible correlation of the predicted causes to environmental changes, for instance a sudden rise in temperature that may contribute to heat rash, or a marked increase in pollen count or poor air quality alerts that may contribute to an allergy response.

Therefore, API data may provide valuable contribution to the predicted diagnosis, especially when Curtis is being evaluated by a remotely located veterinarian that does not have the benefit of understanding the local weather or air quality changes as would a veterinarian located in the same town as Curtis.

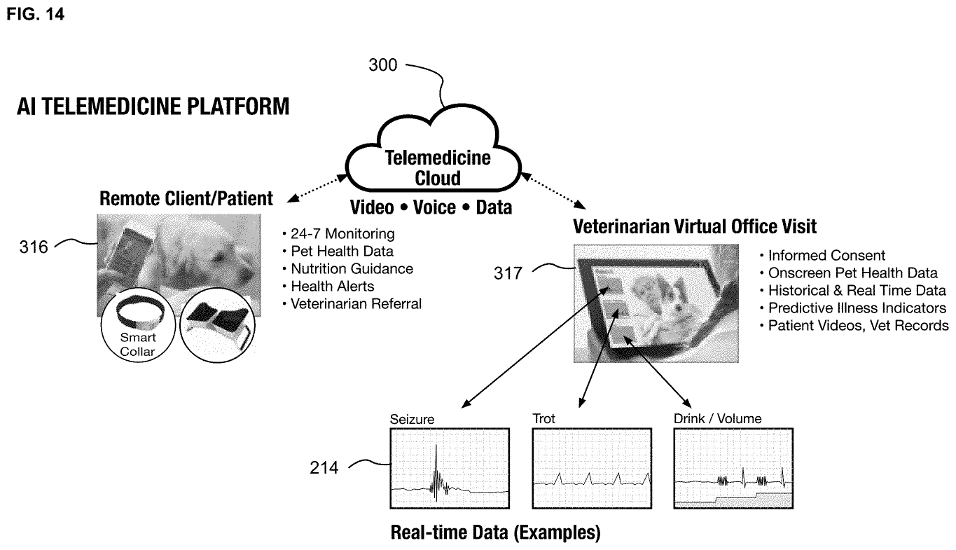

FIG. 14 is an exemplary diagram showing the improved telemedicine platform that connects the veterinarian, client and pet. As previously discussed, the in-home telemedicine ecosystem 316 comprises one or more IoT devices and a smartphone with an installed telemedicine software application. The IoT devices communicate wirelessly with the smartphone app, either directly or indirectly. The in-home ecosystem therefore provides for continuous monitoring of a pet's behaviors, and provides the pet owner with continual updates on the pet's health, and if determined through the cloud-based behavioral analysis, refers or connects the pet owner with a remotely located veterinarian.

On the other hand, the veterinarian may access the telemedicine portal 317 that provides for voice, video and data communication with the pet owner, and further graphically presents relevant behavior signatures 214 to aid in further diagnosis. Many additional features of the veterinarian's portal allow the exporting of the telemedicine examination to the veterinarian's pet health records software systems, and provides for the pet owner to digitally sign any appropriate forms, such as an informed consent form traditionally executed prior to treatment of a new patient.

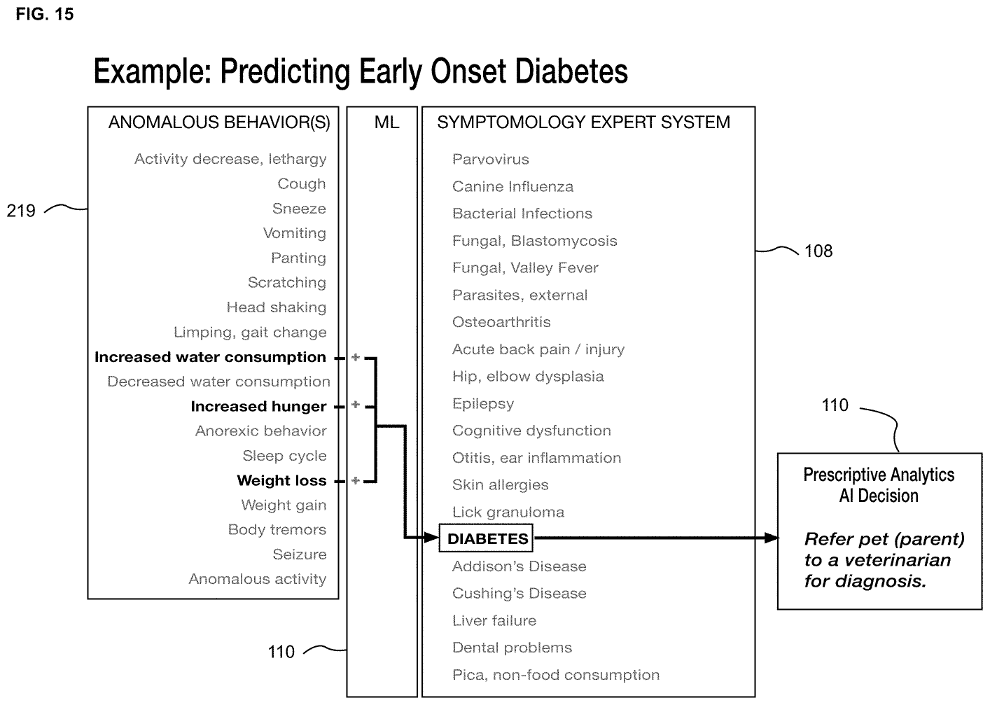

FIG. 15 is an exemplary diagram showing the predictive diagnosis of a disease derived from the analysis of a plurality of animal behaviors.

A listing of derived pet behaviors 219 is shown, the list of behaviors representing a snapshot of hundreds of possible behaviors. More specifically, in the illustrative example of identifying early onset diabetes in a subject animal, anomalous derived data was computed using IoT data from multiple devices. For instance, increased water consumption may be determined by analyzing the animal's average water consumption data from a wireless water bowl as previously described, and ML trend analysis over time determined an anomalous increase.

Increased hunger behavior may be determined by analyzing the animal's average meal consumption time via data from a wireless food bowl as previously described, and ML trend analysis over time indicating a significant decrease in the time it takes to consume the meal—an indicator of increased hunger. Further, behavioral data from the smart collar, and specifically behavioral data that correlates to chewing is analyzed, and the trend analysis may indicate a marked trend increase in chewing behavior, especially outside of mealtime.

Weight loss trend is analyzed using IoT scale data. The pet's weight trend is automatically updated daily as the pet walks across a wireless scale. In the example, the trend indicates anomalous weight loss compared to the pet's historical trend.

A rules based ML engine 110, having been trained to recognize co-occurring anomalous behavior presents the data pattern to a symptomology expert system to identify diabetes as the closest behavior pattern. Prescriptive analysis by a rules based AI engine determines diabetes requires veterinary care and decides to notify the pet owner and veterinarian of the predicted diagnosis, and provides for connecting the pet owner to the veterinarian via a voice, video and data telemedicine platform.

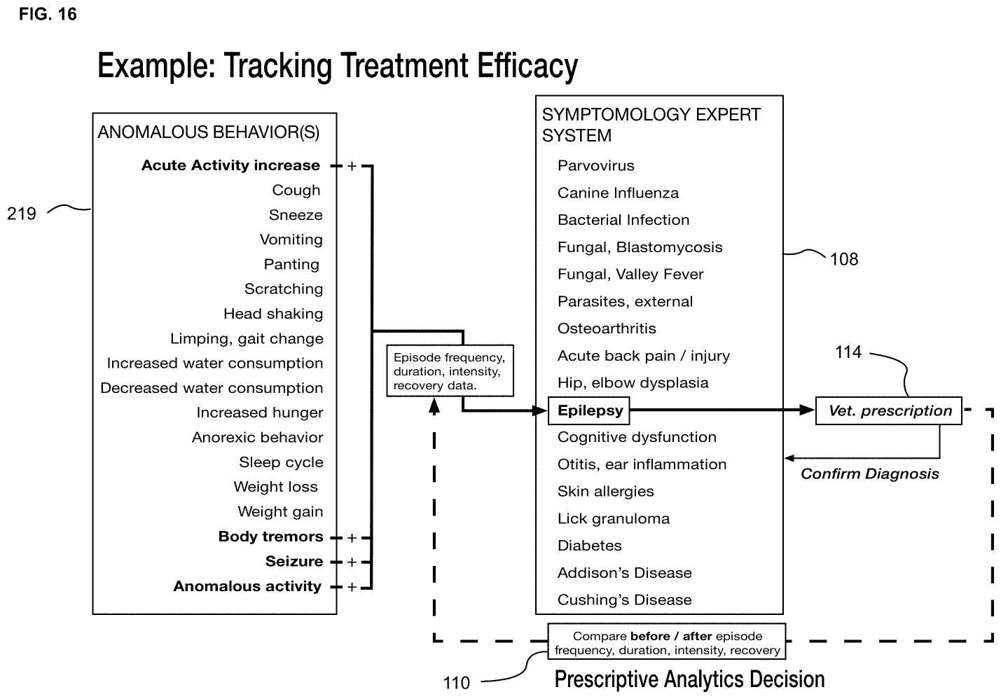

FIG. 16 is an exemplary diagram showing a method of remotely evaluating the efficacy of a treatment of an illness derived from the analysis of a plurality of animal behaviors.

A listing of derived pet behaviors 219 is shown, the list of behaviors representing a snapshot of hundreds of possible behaviors. In the example, an ML engine identifies co-occurring behavioral anomalies including acute activity increase, body tremors, seizures and anomalous activity. Based on rules parameters related to the frequency duration, intensity and recovery match the pattern to the symptomology expert system resulting in a predictive diagnosis of epilepsy. Prescriptive analysis by a rules based AI engine determines epilepsy requires veterinary care and connects the pet to the veterinarian via the telemedicine platform. In the drawing, a veterinarian (1) confirms the predicted diagnosis thereby validating the ML rules analysis and illness prediction, and (2) prescribes a medication to reduce the occurrence or intensity of epileptic episodes.

The addition of a treatment to the pet's record, and specifically a treatment targeting the co-occurring anomalous behavior generates a new rule to provide comparative analysis of the symptomology at future occurrences of the co-occurring anomalies to the first recorded pattern of the co-occurring anomalies, and provide that comparative data to the veterinarian to assess the efficacy of the prescribed treatment, thereby allowing the veterinarian to adjust the treatment protocol as may be appropriate.

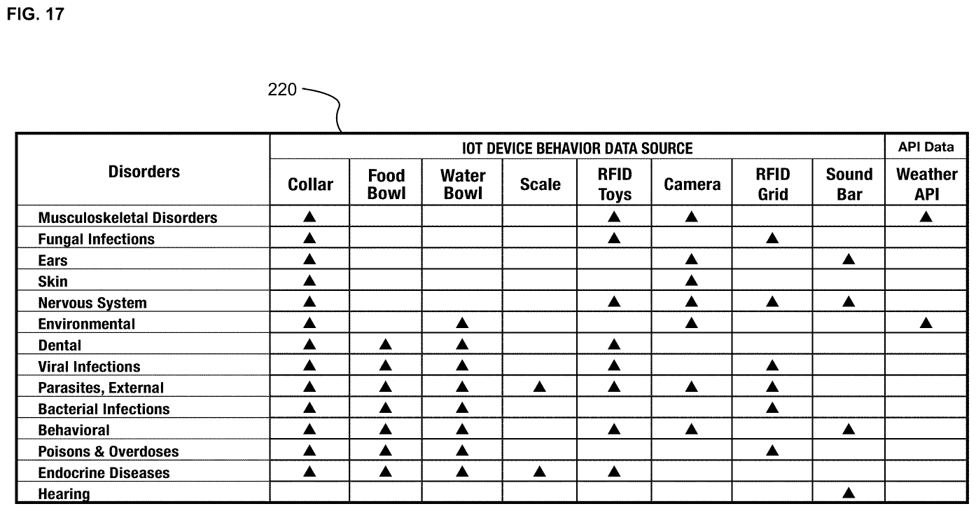

FIG. 17 is an exemplary diagram showing a list of animal disorders and examples of the corresponding data acquisition means used to predictively identify the disorders. More specifically, a disorders chart 220 lists in the first column various top level disorders encountered by pets. It should be noted that within these top-level disorders, there are innumerable sub-level disorders not shown because doing so would be overwhelming and distract from the present description. It is the objective of a veterinary telemedicine platform to identify the occurrence and/or onset of all of these disorders as a means to maintain better pet health, or to cause the pet owner to engage a veterinarian to treat the disorders at the earliest possible opportunity.

A row of IoT devices is shown including a smart collar, food bowl, water bowl, and other IoT devices as previously described herein. As can be seen, data from a smart collar is used in predictively diagnosing most of the disorders. However, as previously described, the analysis of behavioral data from additional IoT devices provides a rules-based engine to more accurately triage the disorders by identifying and analyzing co-occurring anomalies. Therefore, moving left to right, data from each additional IoT device marked by a triangle adds important data to the predictive diagnostic analysis. Therefore, a robustness of a telemedicine platform of the present invention increases with the additional data acquired from an increasing number of IoT devices.

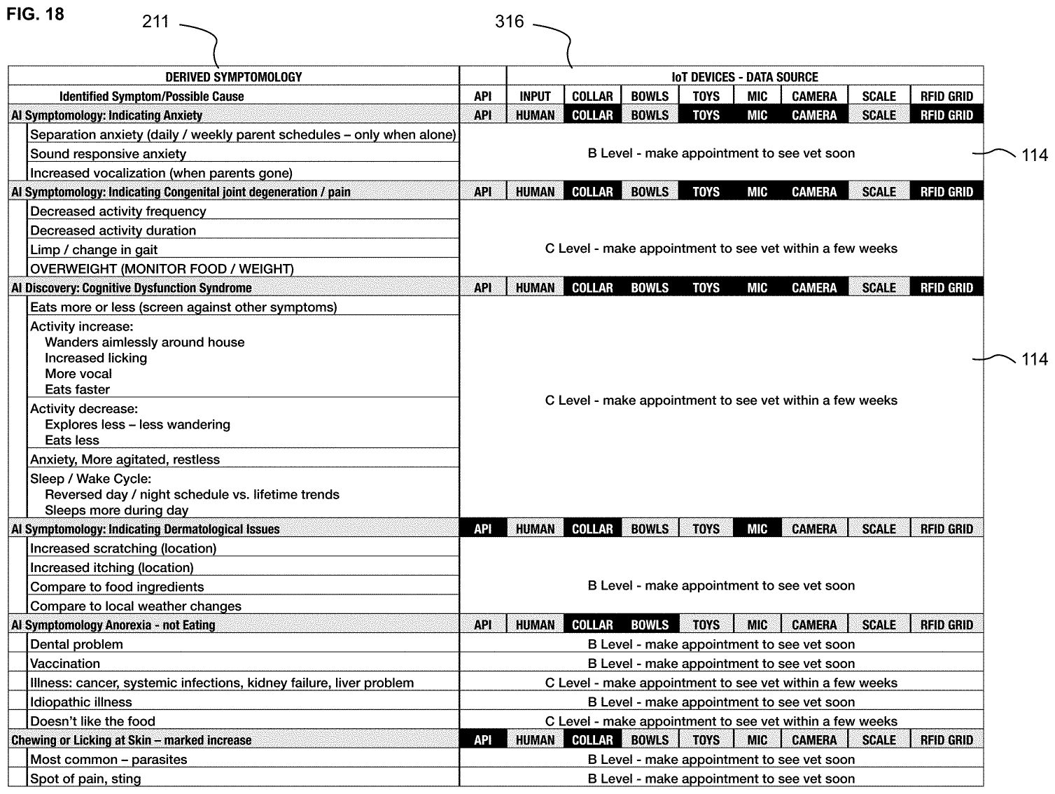

FIG. 18 is an exemplary diagram showing a portion of an expert system.

More specifically, a first column of derived symptomology 211 lists in the shaded cells various illnesses or health conditions that may be experienced by an animal. Subordinate to each shaded cell is a list of co-occurring behaviors that are determined via machine analysis of multiple IoT and/or API data sources as previously described. The subordinate cells also illustrate the large number of conditions and behaviors evaluated to property triage the symptoms to ultimately determine the most likely predicted diagnosis.

A second column shows a plurality of data sources including IoT devices of an in-home telemedicine ecosystem 316. The data sources shown in white text on black background indicate the data sources preferably used to determine the corresponding symptomology in the left column, although more or fewer data sources may nevertheless yield a similar predicted diagnosis.

Further, it is well understood that some acute illnesses require immediate care, such as poisoning, while other chronic or slowly progressing illnesses or diseases that still require veterinary care may reasonably wait days or weeks to schedule a virtual or in-office veterinary appointment.

Therefore, one important feature of the telemedicine platform described above is a means of communicating the level of urgency that the pet owner should seek veterinary care. An urgency of care expert system 114 now shown correlates each predictive diagnosis of the symptomology expert system to a standardized level of urgency with which the pet owner should seek veterinary care, ranging from acute injury requiring immediate emergency care, to the other extreme that may be simply advisory in nature providing instructions to the pet owner to look for additional indications of the illness and seek veterinary care at their discretion.

Prescriptive analysis of the predicted diagnosis triggers an autonomous alert advising the pet owner and/or veterinarian of the predicted diagnosis as well as the level of urgency that treatment of the predictively diagnosed condition warrants.

Unless otherwise defined, all technical and scientific terms used herein have the same meaning as commonly understood by one of ordinary skill in the art to which this invention belongs. Although methods and materials similar to or equivalent to those described herein can be used in the practice or testing of the veterinary telemedicine system and method, suitable methods and materials are described above. All publications, patent applications, patents, and other references mentioned herein are incorporated by reference in their entirety to the extent allowed by applicable law and regulations. The veterinary telemedicine system and method may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

What is claimed is:

1. A system for predictively diagnosing a medical condition of a pet, the system comprising;
a library of uniquely identifiable pet behaviors exhibited by a plurality of similar pets, each uniquely identifiable pet behavior comprising a uniquely identifiable data signature associated with the behavior, the library of uniquely identifiable pet behaviors stored in a cloud database;
a library of known symptoms associated with each of a plurality of medical conditions of a pet, the library of known symptoms stored in the cloud database;
a plurality of individual pet health records stored in the cloud database, each record comprising at least uniquely identifiable physiological and behavior data;
a local computer adapted to receive a set of data from a plurality of sensors associated with a unique pet and further adapted to transmit the set of data to an individual pet health record associated with the unique pet in the cloud database;
wherein the cloud database is adapted to perform a rules-based machine analysis of a pet's physiological and behavior data and the identification of anomalies related to physiological or behavioral changes compared to the unique pet's historical data trends;
wherein the cloud database is adapted to perform a rules-based machine analysis of physiological and/or behavior data anomalies that positively correlate with symptoms stored in a symptoms library;
wherein the cloud database is adapted to perform a rules-based machine that correlates the symptoms to one or more predicted medical conditions; and
a rules-based machine that communicates the predicted medical condition to a pet guardian or a veterinarian.

2. The system of claim 1, wherein the plurality of sensors associated with a pet comprises one or more of a wearable smart collar, a metered water bowl, a metered food bowl, a scale, a motion sensing camera, a microphone, or an RFID or NFC tag;

3. The system of claim 1, wherein data from at least one of an accelerometer, a magnetometer or a gyroscope sensor of a wearable smart collar comprises a data signature relating to a uniquely identifiable behavior of the uniquely identifiable pet.

4. The system of claim 1, wherein the data from one or more of a metered water bowl, a metered food bowl, a scale, a motion sensing camera, a microphone, or an RFID or NFC tag is stored in an individual health record of the uniquely identifiable pet.

5. The system of claim 1, wherein data is periodically received from one or more sensors is appended to each pet's health record, with like data being appended to like data thereby creating unique data trends.

6. The system of claim 1, wherein one of the plurality of sensors comprises a water bowl sensor for tracking consumption time and a volume of water consumed over a defined period of time.

7. The system of claim 1, wherein one of the plurality of sensors comprises a food bowl sensor for tracking a time, a duration and a food volume consumed over a defined period of time.

8. The system of claim 1, wherein one of the plurality of sensors comprises a scale for weighing the pet, and wherein data associated with the scale is time-stamped data comprising a weight of the uniquely identifiable pet.

9. The system of claim 1, wherein the system further comprises a motion-sensing camera, a microphone, or an RFID or NFC tag, wherein the data transmitted from the local computer comprises time-stamped data comprising one or more of an activity, a movement, a location or a vocal activity of the pet;
wherein the data transmitted from the local computer correlates to repeatable and/or predictable physiology and behavior of the pet; and
wherein the data transmitted from the local computer is usable to diagnose an illness of the pet.

10. The system of claim 1, wherein the local computer comprises a smartphone or a WiFi router.

11. A method of using the system of claim 1, comprising:
receiving the set of data at the local computer;
transmitting the set of data from the local computer to the cloud database;
storing the set of data as at least part of the data signature in the cloud database; and
using the data signature for machine identification of animal illness to diagnose an illness of the pet.

12. A veterinary telemedicine system, comprising:
a cloud database comprising a library of pet behaviors associated with a plurality of pets, each behavior comprising a data signature associated with the behavior;
an in-home pet monitoring system adapted to communicate with the cloud database to provide data regarding an individual pet's behavior to the individual pet's historical data record; and
a veterinary telemedicine portal adapted to communicate with the cloud database to provide a veterinarian with access to the cloud database;
wherein the cloud database comprises a machine learning capability to identify a predicted illness of the pet using the library of pet behaviors or using a library of symptoms that correlate to a library of medical conditions.

13. The veterinary telemedicine system of claim 12, wherein the in-home pet monitoring system comprises a plurality of pet wearable, transportable, or fixed IoT devices adapted to communicate data regarding pet behaviors to the cloud database.

14. The veterinary telemedicine system of claim 12, wherein the library comprises a pet behavior database associated with the individual pet, and wherein the library further comprises data received from the plurality of IoT devices, and wherein the cloud database comprises:
a plurality of rules-based engines that analyze data signatures and correlate data signatures in the library to behavior patterns in the library; and
a symptomology expert system.

15. The system of claim 12, wherein the veterinary telemedicine portal provides a veterinarian with voice, video, and data communication with a remotely located pet owner via the in-home pet monitoring system.

16. A method of using the system of claim 12, comprising:
receiving data at the in-home pet monitoring system from at least two IoT devices;
transmitting data from the in-home pet monitoring system to the cloud database;
storing the set of data as at least part of the data signature in the cloud database;
using the data signature to generate a machine identification of an animal illness to diagnose an illness of the pet based on co-occurring anomalous pet behavior data from the at least three IoT devices; and
providing an alert to a pet owner to take action in response to the illness.

17. A system for creating a library of pet behaviors associated with an individual pet, each behavior comprising a data signature associated with the behavior, the system comprising:
a plurality of sensors comprising a water bowl sensor, a food bowl sensor, a camera, a scale, a microphone, a wearable smart collar comprising at least one of an accelerometer, a gyroscope, or a magnetometer, and/an RFID or NFC tag;
a smartphone or a WiFi router adapted to receive a set of data from the plurality of sensors and further adapted to transmit the set of data to a cloud database;
wherein the data signature comprises data relating to a motion of the pet;
wherein the data signature comprises a weight of the pet;
wherein the data signature comprises a stop and stop time of the pet's food and water consumption; and
wherein the library of pet behaviors is stored on the cloud database.
